SEQUENCE LISTING (4 sequences)

SEQ ID NO: 1 - protein, 8 aa, Unknown
  source: /note=“Description of Unknown Bacterial LOV domain sequence”
  MOD_RES (2)..(2): Any amino acid
GXNCRFLQ

SEQ ID NO: 2 - protein, 8 aa, Unknown
  source: /note=“Description of Unknown Bacterial LOV sub-motif sequence”
  MOD_RES (2)..(2): Any amino acid
  VARIANT (3)..(3): /replace=“Asp”
  VARIANT (5)..(5): /replace=“His”
  VARIANT (6)..(6): /replace=“Ile”
  VARIANT (8)..(8): /replace=“Ala”
  SITE (1)..(8): /note=“Variant residues given in the sequence have no preference with respect to those in the annotations for variant positions”
GXNCRFLQ

SEQ ID NO: 3 - protein, 7 aa, Unknown
  source: /note=“Description of Unknown Bacterial LOV sub-motif sequence”
  MOD_RES (2)..(4): Any amino acid
  VARIANT (6)..(6): /replace=“Glu”
  SITE (1)..(7): /note=“Variant residues given in the sequence have no preference with respect to those in the annotations for variant positions”
FXXXTGY

SEQ ID NO: 4 - protein, 11 aa, Unknown
  source: /note=“Description of Unknown Bacterial LOV sub-motif sequence”
  VARIANT (2)..(2): /replace=“Phe”
  MOD_RES (3)..(5): Any amino acid
  VARIANT (6)..(6): /replace=“Asp”
  MOD_RES (7)..(8): Any amino acid
  VARIANT (9)..(9): /replace=“Leu”
  MOD_RES (10)..(10): Any amino acid
  SITE (1)..(11): /note=“Variant residues given in the sequence have no preference with respect to those in the annotations for variant positions”
NYXXXGXXFXN

(19) United States
(12) Patent Application Publication
(10) Pub. No.: US 2021/0204501 A1
(43) Pub. Date: Jul. 8, 2021

(54) COMPOSITIONS AND METHODS FOR INCREASING PLANT YIELD

(71) Applicants: THE REGENTS OF THE UNIVERSITY OF CALIFORNIA, Oakland, CA (US); CARNEGIE INSTITUTION OF WASHINGTON, Washington, DC (US)

(72) Inventors: Winslow BRIGGS, Washington, DC (US); Roberto BOGOMOLNI, Santa Cruz, CA (US); Tong-Seung TSENG, Washington, DC (US); Rajnish KHANNA, Washington, DC (US)

(73) Assignees: THE REGENTS OF THE UNIVERSITY OF CALIFORNIA, Oakland, CA (US); CARNEGIE INSTITUTION OF WASHINGTON, Washington, DC (US)

(21) Appl. No.: 17/058,607

(22) PCT Filed: May 24, 2019
(86) PCT No.: PCT/US2019/034025

§ 371 Date: Nov. 24, 2020

Related U.S. Application Data
(60) Provisional application No. 62/675,892, filed on May 24, 2018.

(51) Int. Cl. A01H 3/02; C12N 1/20; C12N 13/00

(52) U.S. Cl. CPC A01H 3/02; C12N 13/00; C12N 1/20

(57) ABSTRACT

The disclosure provides compositions and methods for infecting a legume plant and/or increasing the yield of a legume plant by providing a population of light-activated, nitrogen-fixing bacteria by illuminating a population of nitrogen-fixing bacteria with a blue light and delivering to the legume plant the population of light-activated, nitrogen-fixing bacteria.

### SEQUENCE LISTING

The instant application contains a Sequence Listing which has been submitted electronically in ASCII format and is hereby incorporated by reference in its entirety. Said ASCII copy, created on May 22, 2019, is named 102913-001010WO-1139039_SL.txt and is 2,719 bytes in size.

### BACKGROUND

The total value of legume crops today is around 100 billion dollars in the U.S., 60 billion dollars in Brazil, and 40 billion dollars in Argentina. A modest improvement in crop yield could increase by billions of dollars the commercial value of legume crops in the U.S. and abroad. Innovative methods for cultivating crops and improving crop yield are needed.

### SUMMARY

In one aspect, the disclosure features a method of infecting a legume plant with a population of light-activated, nitrogen-fixing bacteria, comprising: (a) providing a population of light-activated, nitrogen-fixing bacteria by illuminating a population of nitrogen-fixing bacteria with a light having a wavelength of between 350 and 750 nm and an intensity of between 0.1 and 200 μmol·m²·s⁻¹ for a period of between 1 second and 24 hours, and (b) delivering to the legume plant the population of light-activated, nitrogen-fixing bacteria, wherein the legume plant comprises a root with functional root hairs and wherein the population of light-activated, nitrogen-fixing bacteria infects the root of the legume plant.

In another aspect, the disclosure features a method of increasing yield of a legume plant, comprising: (a) providing a population of light-activated, nitrogen-fixing bacteria by illuminating a population of nitrogen-fixing bacteria with a light having a wavelength of between 350 and 750 nm and an intensity of between 0.1 and 200 μmol·m²·s⁻¹ for a period of between 1 second and 24 hours, and (b) delivering to the legume plant the population of light-activated, nitrogen-fixing bacteria, wherein the legume plant comprises a root with functional root hairs, wherein the population of light-activated, nitrogen-fixing bacteria infects the root of the legume plant, and wherein the yield of the legume plant is at least 6% greater than the yield of a legume plant that is infected by a population of nitrogen-fixing bacteria that is not light activated.

In some embodiments of the previous two aspects, step (b) comprises delivering the light-activated, nitrogen-fixing bacteria to the legume plant through an irrigation system (e.g., a drip irrigation system).

In some embodiments of the first aspect, the yield of the legume plant is at least 6% greater than the yield of a legume plant that is infected by a population of nitrogen-fixing bacteria that is not light activated.

In some embodiments, the method results in a greater number of nodules containing leghemoglobin formed on the root of the legume plant compared to the number of nodules containing leghemoglobin formed on the root of a legume plant that is infected by a population of nitrogen-fixing bacteria that is not light activated. In some embodiments, the method results in a greater number of leghemoglobin per nodule on the root of the legume plant compared to the number of leghemoglobin per nodule on the root of a legume plant that is infected by a population of nitrogen-fixing bacteria that is not light activated.

In some embodiments of the previous two aspects, the population of nitrogen-fixing bacteria are in a genus selected from the group consisting of Allorhizobiaum, Aminobacter, Azorhizobium, Bradyrhizobium, Devosia, Ensifer, Mesorhizobium, Methylobacterium, Microvirga, Ochrobactrum, Phyllobacterium, Rhizobium, Shinella, or Sinorhizobium.

In particular embodiments, the population of nitrogen-fixing bacteria are in the genus Rhizobium (e.g., R. aggregatum, R. alamii, R. alkalisoli, R. borbori, R. cellulosilyticum, R.cireri, R. daejeonense, R. endophyticum, R. etli, R. fabae, R. fredii, R. galegae, R. gallicum, R. giardinii, R. hainanense, R. halophytocola, R. herbae, R. huakuii, R. huautlense, R. indigoferae, R. japonicum, R. larrymoorei, R. leguminosarum, R. leucaenae, R. loessense, R. loti, R. lupini, R. lusitanum, R. mediterraneum, R. melioti, R. misosinicum, R. miluonense, R. mongolense, R. multihospitium, R. oryze, R. petrolearium, R. phaseoli, R. pisi, R. pseudoryzae, R. pusense, R. radiobacter, R. rhizogenese, R. rosettiformans, R. rubi, R. selenitireducens, R. skierniewicense, R. soli, R. sullae, R. taibaishanense, R. tianshanense, R. tibeticum, R. trifolii, R. sphaerophysae, R. tropici, R. grahamii, R. mesoameicanum, R. nepotum, R. tubonense, R. undicola, R. vallis, R. vignae, R. vitis, or R. yanglingense). In particular embodiments, the population of nitrogen-fixing bacteria are R. leguminosarum.

In other embodiments, the population of nitrogen-fixing bacteria are in the genus Bradyrhizobium (e.g., Bradyrhizobium japonicum) or Sinorhizobium (e.g., Sinorhizobium meliloti).

In another aspect, the disclosure features a method of infecting a legume plant with a light-activated, nitrogen-fixing Rhizobium culture, the method comprising: (a) activating a nitrogen-fixing Rhizobium culture by illuminating the nitrogen-fixing Rhizobium culture with a light having a wavelength of between 350 and 750 nm and an intensity of between 0.1 and 200 μmol·m²·s⁻¹ for a period of between 1 second and 24 hours, thereby creating a light-activated, nitrogen-fixing Rhizobium culture; and (b) contacting a legume plant seed with the light-activated, nitrogen-fixing Rhizobium culture after the legume plant seed has developed at least one functional root hair.

In some embodiments of the methods described herein, the population of nitrogen-fixing bacteria is illuminated with a light having a wavelength of, e.g., between 350 and 700 nm, between 350 and 650 nm, between 350 and 600 nm, between 350 and 550 nm, between 350 and 500 nm, between 350 and 450 nm, between 350 and 400 nm, between 400 and 750 nm, between 450 and 750 nm, between 500 and 750 nm, between 550 and 750 nm, between 600 and 750 nm, between 650 and 750 nm, between 700 and 750 nm, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, or 750 nm.

In some embodiments of the methods described herein, the population of nitrogen-fixing bacteria is illuminated with a light having a wavelength of between 400 and 500 nm (e.g., between 445 and 455 nm). In particular embodiments, the wavelength is 450 nm.

In some embodiments of the methods described herein, the population of nitrogen-fixing bacteria is illuminated with a light having an intensity of, e.g., between 0.1 and 150 μmol·m²·s⁻¹, between 0.1 and 100 μmol·m²·s⁻¹, between 0.1 and 50 μmol·m²·s⁻¹, between 0.1 and 10 μmol·m²·s⁻¹, between 0.1 and 1 μmol·m²·s⁻¹, between 0.1 and 0.5 μmol·m²·s⁻¹, between 0.5 and 200 μmol·m²·s⁻¹, between 1 and 200 μmol·m²·s⁻¹, between 10 and 200 μmol·m²·s⁻¹, between 50 and 200 μmol·m⁻²·s⁻¹, between 100 and 200 μmol·m⁻²·s⁻¹, or between 150 and 200 μmol·m²·s⁻¹, for a period of between 1 second and 24 hours (e.g., between 1 second and 20 hours, between 1 second and 15 hours, between 1 second and 10 hours, between 1 second and 5 hours, between 1 second and 1 hour, between 1 second and 30 minutes, between 1 second and 20 minutes, between 1 second and 10 minutes, between 1 second and 1 minute, between 1 second and 30 seconds, between 1 second and 10 seconds, between 1 second and 5 seconds, between 30 seconds and 24 hours, between 1 minute and 24 hours, between 10 minutes and 24 hours, between 24 minutes and 24 hours, between 30 minutes and 24 hours, between 1 hour and 24 hours, between 5 hours and 24 hours, between 10 hours and 24 hours, between 15 hours and 24 hours, or between 20 hours and 24 hours).

In some embodiments of the methods described herein, the nitrogen-fixing bacteria comprise a light, oxygen, and voltage (LOV) domain. The nitrogen-fixing bacteria may naturally express the LOV domain. In other embodiments, the nitrogen-fixing bacteria may be engineered to express the LOV domain.

In some embodiments of the methods described herein, the nitrogen-fixing Rhizobium culture is illuminated with an LED light.

In some embodiments of the methods described herein, step (b) occurs at least 24 hours (e.g., at least 36 hours, at least 48 hours, at least 60 hours, at least 72 hours, at least 84 hours, at least 96 hours, or at least 108 hours, at least 120 hours, at least 132 hours, or at least 144 hours) after the legume plant seed has been planted.

In some embodiments of the methods described herein, step (b) comprises providing the nitrogen-fixing Rhizobium culture to the legume plant seed via an irrigation system (e.g., a drip irrigation).

In some embodiments of the methods described herein, the legume plant is selected from the group consisting of peas, soybeans, alfalfa, clover, vetch, mung bean, vigna, cowpea, trefoil, lupine, peanuts, fava beans, chickpeas, lentils, lupin beans, mesquite, carob, and tamarind.

In another aspect, the disclosure features a device that generates a light-activated, nitrogen-fixing Rhizobium culture and delivers the light-activated Rhizobium culture to a legume plant, the device comprising: (a) a light source configured to provide light to a nitrogen-fixing Rhizobium culture at a wavelength of between 350 and 750 nm and an intensity of between 0.1 and 200 μmol·m²·s⁻¹ for a period of between 1 second and 24 hours; and (b) a delivery system configured to provide the light-activated, nitrogen-fixing Rhizobium culture to the legume plant subsequent to the activation of the nitrogen-fixing Rhizobium culture by the light source in step (a).

In some embodiments of the device, the light source may provide a light having a wavelength of between 400 and 500 nm (e.g., between 445 and 455 nm). In particular embodiments, the wavelength is 450 nm.

In some embodiments of the device, the delivery system comprises an irrigation system (e.g., a drip irrigation system).

In yet another aspect, the disclosure features an inoculant composition comprising a population of light-activated, nitrogen-fixing bacteria previously exposed to light at a wavelength of between 350 and 750 nm and an intensity of between 0.1 and 200 μmol·m²·s⁻¹ for a period of between 1 second and 24 hours. In some embodiments of the inoculant composition, the population of light-activated, nitrogen-fixing bacteria are Bradyrhizobium japonicum, Rhizobium leguminosarum, Sinorhizobium meliloti, or Rhizobium trifolii.

In some embodiments of the inoculant composition, the light-activated, nitrogen-fixing bacteria are engineered to express a LOV domain. In some embodiments, the inoculant composition may be in a liquid or particulate form.

### Definitions

As used herein, the term “nitrogen-fixing bacteria” refers to bacteria that are capable of transforming atmospheric nitrogen into fixed nitrogen, i.e., inorganic compounds containing nitrogen, usable by plants. Nitrogen-fixing bacteria may be wild-type bacteria or engineered bacteria.

As used herein, the term “light-activated, nitrogen-fixing bacteria” refers to bacteria that express a photoreceptor (e.g., a LOV domain) which can be activated by light (e.g., blue light having a wavelength of between 445 and 455 nm (e.g., 450 nm)). Light activation of the bacteria may enhance the capacity of the bacteria to infect legume plants and consequently improve legume agriculture. In some embodiments, the nitrogen-fixing bacteria may naturally express the photoreceptor (e.g., a LOV domain). In some embodiments, the nitrogen-fixing bacteria may be engineered to express the photoreceptor (e.g., a LOV domain).

As used herein, the term “engineered bacteria” refers to bacteria that have been genetically altered. For example, nitrogen-fixing bacteria may be engineered to express a photoreceptor (e.g., a LOV domain).

As used herein, the term “functional root hairs” refers to root hairs developed by the legume plants after the plants have germinated that can be infected by inoculated bacteria.

As used herein, the term “nodules” refers to the small nodes that develop on the roots of legume plants. Within the nodules, nitrogen-fixing bacteria convert nitrogen gas from the atmosphere to ammonia, which can then be assimilated into amino acids, nucleotides, and other cellular constituents for the plants to use. Legume nodules also harbor leghemoglobin, an iron-containing protein that facilitates the diffusion of oxygen. High leghemoglobin content in the nodules may indicate high nitrogen fixation rate or efficiency since nitrogen fixation in the nodules is oxygen sensitive.

### BRIEF DESCRIPTION OF THE DRAWINGS

FIGS. 1A and 1B are bar graphs showing the stem height (FIG. 1A) and stem height difference (FIG. 1B) of fava bean plants after Rhizobium leguminosarum inoculations.

FIG. 2A is a bar graph showing the nodulation numbers of five to six week old fava bean plants that were inoculated with Rhizobium leguminosarum cells either irradiated with blue light or kept in the dark at six days after the seeds were planted.

FIG. 2B is a bar graph showing a comparison of the nodule numbers of fava beans that were inoculated with Rhizobium leguminosarum cells at 0 days following seed planting and at 6 days following seed planting.

FIGS. 3A-3C are bar graphs showing that fava bean seed yield increased when the plants were inoculated with Rhizobium leguminosarum cells irradiated with blue light either at 0 days following seed planting and at 6 days following seed planting.

FIG. 4 is a photograph showing the yields of fava beans that had different inoculation conditions.

FIGS. 5 and 6 are photographs showing the difference of functional nodule formation on plants inoculated with Rhizobium leguminosarum cells irradiated with blue light and Rhizobium leguminosarum cells kept in the dark.

FIG. 7 is a bar graph showing that plants inoculated with bacteria exposed to blue light and watered 4 days prior the inoculation showed higher production of pink nodules than plants inoculated with bacteria exposed to no light (dark condition).

FIG. 8 is a scatterplot showing that plants treated with the bacteria exposed to blue light showed a higher production of mature pods than the plants treated with the bacteria exposed to continuous dark.

FIG. 9 is a scatterplot showing that plants treated with the bacteria exposed to blue light showed a higher production of peas than the plants treated with the bacteria exposed to continuous dark.

FIG. 10 includes photographs showing that 8 principal nodules on a root system inoculated with bacteria treated under blue light condition before (left) and after (right) the nodules were cut. The photograph on the right shows that 2 of the 8 nodules were white (1) and 6 of the 8 nodules were pink (2), indicating that they were functional nodules containing leghemoglobin.

FIG. 11 includes photographs showing that plants inoculated with light-activated bacteria Rhizobium produced more peas per pod on average.

FIG. 12 includes photographs showing that plants treated with light-activated Rhizobium displayed higher chlorophyll production compared to plants treated with Rhizobium left in the dark.

FIG. 13 is a bar graph showing an average per plant of the portions of pods aborted (1), harvested (2), and still maturing (3) at the end of 8 weeks of growth period.

FIG. 14 is a bar graph showing the projected weights of peas produced per acre for peas inoculated with bacteria treated under blue light condition and for peas inoculated with bacteria kept under the dark.

### DETAILED DESCRIPTION OF THE EMBODIMENTS

### I. Introduction

Nitrogen-fixing bacteria are microorganisms capable of transforming atmospheric nitrogen into fixed nitrogen, i.e., inorganic compounds containing nitrogen, usable by plants. Rhizobium is one kind of symbiotic, nitrogen-fixing bacteria that invade the root hairs of host plants where they multiply and stimulate formation of root nodules. Within the nodules of the host plant, the nitrogen-fix bacteria convert free nitrogen to ammonia, which the host plant utilizes for its development. The present disclosure is directed to the concept that illumination of Rhizobium could result in an enhancement in the capacity of the bacteria to infect legume plants and consequently improve legume agriculture.

As discussed in detail further herein, the disclosure shows that inoculation with light-treated bacteria led to a significant improvement of plant development over inoculation with non-illuminated bacteria. The disclosure introduces methods to improve the capacity of Rhizobium bacterial cultures to fertilize legume crops, such as fava beans, peas, soybeans, alfalfa, and peanuts. Legume crops are routinely sprayed with Rhizobium bacteria that infect the plants and live inside the plant roots to provide the plants with fertilizing compounds that they produce from nitrogen in the air. In return, the plants provide the bacteria with the nutrients they need to survive. Pre-illumination of these bacteria before inoculation of the bacteria to the growing crop greatly improves their capacity to infect the plant roots. Irrigation of the plant fields with light-activated inoculates significantly improves fertilization and crop yields over yields obtained with bacteria grown and stored in darkness.

Further, delaying the timing of the bacterial inoculum application until a plant has germinated and has generated a root system that the bacteria can infect is also crucial. Current practice is to provide the bacterial inoculums with the plant seeds at planting time before any roots are available for bacterial infection. However, during the several days required for plant germination and root development, any degree of light activation that the bacteria received during day-time planting disappears by a spontaneous process of deactivation that takes only a few hours. In addition, because no roots are available, the inoculated bacteria have to survive several days under low nutrient conditions in the soil and thus, tend to die before they have ab opportunity to infect the plant.

### II. Nitrogen-Fixing Bacteria

The disclosure provides compositions and methods for infecting legume plants (e.g., peas, soybeans, alfalfa, clover, vetch, mung bean, vigna, cowpea, trefoil, lupine, peanuts, fava beans, chickpeas, lentils, lupin beans, mesquite, carob, and tamarind) and/or increasing the yield of legume plants by providing a population of light-activated, nitrogen-fixing bacteria. Nitrogen-fixing bacteria are microorganisms capable of transforming atmospheric nitrogen into fixed nitrogen, i.e., inorganic compounds containing nitrogen, usable by plants. Rhizobium is one kind of symbiotic, nitrogen-fixing bacteria that invade the root hairs of host plants where they multiply and stimulate formation of root nodules. Within the nodules of the host plant, the nitrogen-fix bacteria convert free nitrogen to ammonia, which the host plant utilizes for its development. In some embodiments, the nitrogen-fixing bacteria used in compositions and methods of the disclosure are in a genus selected from the group consisting of Allorhizobiaum, Aminobacter, Azorhizobium, Bradyrhizobium, Devosia, Ensifer, Mesorhizobium, Methylobacterium, Microvirga, Ochrobactrum, Phyllobacterium, Rhizobium, Shinella, or Sinorhizobium. In particular embodiments, the population of nitrogen-fixing bacteria are in the genus Rhizobium (e.g., R. aggregatum, R. alamii, R. alkalisoli, R. borbori, R. cellulosilyticum, R.cireri, R. daejeonense, R. endophyticum, R. etli, R. fabae, R. fredii, R. galegae, R. gallicum, R. giardinii, R. hainanense, R. halophytocola, R. herbae, R. huakuii, R. huautlense, R. indigoferae, R. japonicum, R. larrymoorei, R. leguminosarum, R. leucaenae, R. loessense, R. loti, R. lupini, R. lusitanum, R. mediterraneum, R. melioti, R. misosinicum, R. miluonense, R. mongolense, R. multihospitium, R. oryze, R. petrolearium, R. phaseoli, R. pisi, R. pseudoryzae, R. pusense, R. radiobacter, R. rhizogenese, R. rosettiformans, R. rubi, R. selenitireducens, R. skierniewicense, R. soli, R. sullae, R. taibaishanense, R. tianshanense, R. tibeticum, R. trifolii, R. sphaerophysae, R. tropici, R. grahamii, R. mesoameicanum, R. nepotum, R. tubonense, R. undicola, R. vallis, R. vignae, R. vitis, or R. yanglingense). In particular embodiments, the population of nitrogen-fixing bacteria are R. leguminosarum. In other embodiments, the population of nitrogen-fixing bacteria may be Bradyrhizobium japonicum or Sinorhizobium meliloti.

The nitrogen-fixing bacteria may be a naturally existing (i.e., wild-type) bacteria or an engineered bacteria. The nitrogen-fixing bacteria may contain photoreceptors that sense the wavelength and intensity of light and convert the light into chemical energy. Examples of bacterial photoreceptors include, but are not limited to, phytochrome domains, light, oxygen, and voltage (LOV) domains, blue-light photoreceptor (BLUF) domains, photoactive yellow proteins, and sensory and light-harvesting rhodopsins. In some embodiments, the nitrogen-fixing bacteria used in the compositions and methods of the disclosure comprise a LOV domain. LOV domains are a subset of the large and diverse PAS superfamily, which are implicated in cellular signaling processes across all kingdoms of life. A bacterial LOV domain may comprise three domains: a LOV domain at the N-terminus (the sensory domain), a histidine kinase (HK) at the C-terminus (the output domain), and a PAS domain between them. Examples of LOV domains are known in the art. A bacterial LOV domain may comprise the sequence of GXNCRFLQ (SEQ ID NO:1). Two highly conserved motifs of 43 and 48 amino acids in length have been reported (Glantz et al., 2016, PNAS 113: E1442-E1451) based upon projecting the flavin-binding pocket onto the 3D structure of Avena sativa LOV2. Several sub-motifs have also been reported, for example, GX(N/D)C(R/H)(F/I)L(Q/A) (SEQ ID NO:2), FXXXT(G/E)Y (SEQ ID NO:3), and N(Y/F)XXX(G/D)XX(F/L)XN (SEQ ID NO:4), which are required for blue light sensitivity. In particular embodiments of the disclosure, the compositions and methods described herein provide a population of light-activated, nitrogen-fixing Rhizobium leguminosarum that contain a LOV domain. In particular embodiments of the disclosure, the compositions and methods described herein provide a population of light-activated, nitrogen-fixing, wild-type Rhizobium leguminosarum.

In some embodiments, the population of nitrogen-fixing bacteria are wild-type bacteria containing a LOV domain. In some embodiments, the population of nitrogen-fixing bacteria are engineered to express a LOV domain. Methods to engineer bacteria to express certain desired proteins are available in the art and discussed in detail further herein.

Light-Activated, Nitrogen-Fixing Bacteria

A population of light-activated, nitrogen-fixing bacteria may be generated by illuminating or irradiating a population of nitrogen-fixing bacteria (e.g., a population of nitrogen-fixing bacteria that had been kept in the dark) with a light having a wavelength of between 350 and 750 nm (e.g., between 350 and 700 nm, between 350 and 650 nm, between 350 and 600 nm, between 350 and 550 nm, between 350 and 500 nm, between 350 and 450 nm, between 350 and 400 nm, between 400 and 750 nm, between 450 and 750 nm, between 500 and 750 nm, between 550 and 750 nm, between 600 and 750 nm, between 650 and 750 nm, between 700 and 750 nm, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, or 750 nm). In some embodiments, the bacteria may be illuminated with a light having a wavelength of between 400 and 500 nm (e.g., between 410 and 500 nm, between 420 and 500 nm, between 430 and 500 nm, between 440 and 500 nm, between 450 and 500 nm, between 460 and 500 nm, between 470 and 500 nm, between 480 and 500 nm, between 490 and 500 nm, between 400 and 490 nm, between 400 and 480 nm, between 400 and 470 nm, between 400 and 460 nm, between 400 and 450 nm, between 400 and 440 nm, between 400 and 430 nm, between 400 and 420 nm, or between 400 and 410 nm). In particular embodiments, the bacteria may be illuminated with a light having a wavelength of between 445 and 455 nm (e.g., 445, 446, 447, 448, 449, 450, 451, 452, 453, 454, or 455 nm). In particular embodiments, the bacteria may be illuminated with a light having a wavelength of 450 nm.

The intensity and duration of light (e.g., light having a wavelength of between 445 and 455 nm, e.g., 450 nm) used to generate the population of light-activated, nitrogen-fixing bacteria may be between 0.1 and 200 μmol·m²·s⁻¹ (e.g., between 0.1 and 150 μmol·m⁻²·s⁻¹, between 0.1 and 100 μmol·m⁻²·s⁻¹, between 0.1 and 50 μmol·m⁻²·s⁻¹, between 0.1 and 10 μmol·m⁻²·s⁻¹, between 0.1 and 1 μmol·m⁻²·s⁻¹, between 0.1 and 0.5 μmol·m⁻²·s⁻¹, between 0.5 and 200 μmol·m⁻²·s⁻¹, between 1 and 200 μmol·m⁻²·s⁻¹, between 10 and 200 μmol·m⁻²·s⁻¹, between 50 and 200 μmol·m⁻²·s⁻¹, between 100 and 200 μmol·m⁻²·s⁻¹, or between 150 and 200 μmol·m⁻²·s⁻¹) for a period of between 1 second and 24 hours (e.g., between 1 second and 20 hours, between 1 second and 15 hours, between 1 second and 10 hours, between 1 second and 5 hours, between 1 second and 1 hour, between 1 second and 30 minutes, between 1 second and 20 minutes, between 1 second and 10 minutes, between 1 second and 1 minute, between 1 second and 30 seconds, between 1 second and 10 seconds, between 1 second and 5 seconds, between 30 seconds and 24 hours, between 1 minute and 24 hours, between 10 minutes and 24 hours, between 24 minutes and 24 hours, between 30 minutes and 24 hours, between 1 hour and 24 hours, between 5 hours and 24 hours, between 10 hours and 24 hours, between 15 hours and 24 hours, or between 20 hours and 24 hours).

### III. Inoculant Compositions

The disclosure also provides inoculant compositions that contain a population of light-activated, nitrogen-fixing bacteria (e.g., Rhizobium leguminosarum) that can be used in methods of infecting legume plants and methods of increasing the yield of legume plants. The population of light-activated, nitrogen-fixing bacteria in the inoculant composition may be Bradyrhizobium japonicum, Rhizobium leguminosarum, Sinorhizobium meliloti, or Rhizobium trifolii. In particular embodiments, the population of light-activated, nitrogen-fixing bacteria in the inoculant composition may be Rhizobium leguminosarum. The bacteria in the inoculant composition may naturally express a LOV domain or may be engineered to express a LOV domain. The population of light-activated, nitrogen-fixing bacteria in the inoculant composition may be made by illuminating a population of nitrogen-fixing bacteria (e.g., Rhizobium leguminosarum) with a light having a wavelength of between 350 and 750 nm (e.g., between 350 and 700 nm, between 350 and 650 nm, between 350 and 600 nm, between 350 and 550 nm, between 350 and 500 nm, between 350 and 450 nm, between 350 and 400 nm, between 400 and 750 nm, between 450 and 750 nm, between 500 and 750 nm, between 550 and 750 nm, between 600 and 750 nm, between 650 and 750 nm, between 700 and 750 nm, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, or 750 nm) and an intensity of between 0.1 and 200 μmol·m⁻²·s⁻¹ (e.g., between 0.1 and 150 μmol·m² s⁻¹, between 0.1 and 100 μmol·m² s⁻¹, between 0.1 and 50 μmol·m⁻²·s⁻¹, between 0.1 and 10 μmol·m⁻²·s⁻¹, between 0.1 and 1 μmol·m⁻²·s⁻¹, between 0.1 and 0.5 μmol·m⁻²·s⁻¹, between 0.5 and 200 μmol·m⁻²·s⁻¹, between 1 and 200 μmol·m⁻²·s⁻¹, between 10 and 200 μmol·m⁻²·s⁻¹, between 50 and 200 μmol·m⁻²·s⁻¹, between 100 and 200 μmol·m⁻²·s⁻¹, or between 150 and 200 μmol·m²·s⁻¹) for a period of between 1 second and 24 hours (e.g., between 1 second and 20 hours, between 1 second and 15 hours, between 1 second and 10 hours, between 1 second and 5 hours, between 1 second and 1 hour, between 1 second and 30 minutes, between 1 second and 20 minutes, between 1 second and 10 minutes, between 1 second and 1 minute, between 1 second and 30 seconds, between 1 second and 10 seconds, between 1 second and 5 seconds, between 30 seconds and 24 hours, between 1 minute and 24 hours, between 10 minutes and 24 hours, between 24 minutes and 24 hours, between 30 minutes and 24 hours, between 1 hour and 24 hours, between 5 hours and 24 hours, between 10 hours and 24 hours, between 15 hours and 24 hours, or between 20 hours and 24 hours). In particular embodiments, the population of light-activated, nitrogen-fixing bacteria in the inoculant composition may be made by illuminating a population of nitrogen-fixing bacteria (e.g., Rhizobium leguminosarum) with a light having a wavelength of between 445 and 455 nm (e.g., 445, 446, 447, 448, 449, 450, 451, 452, 453, 454, or 455 nm; e.g., 450 nm).

Moreover, the inoculant composition containing a population of light-activated, nitrogen-fixing bacteria (e.g., Rhizobium leguminosarum) may be in a liquid or particular form. The composition may be in a suitable form for direct application or as a concentrate of primary composition that requires dilution with a suitable quantity of water or other diluent before application. The concentration of the bacterial inoculates (e.g., the population of light-activated, nitrogen-fixing bacteria) in the inoculant composition may vary depending upon, for example, the nature of the particular formulation, specifically, whether it is a concentrate or to be used directly, and the type of plant. In particular embodiments, the inoculant composition containing a population of light-activated, nitrogen-fixing bacteria (e.g., Rhizobium leguminosarum) may be in a liquid form and delivered to the legume plants through an irrigation system (e.g., a drip irrigation system). In some embodiments, the inoculant composition may be applied to the legume plants during seeding or growth of the plants. In particular embodiments, the inoculant composition described herein is applied to the legume plants a few days (e.g., 1 to 15 days, 1, 2, 3, 4, 5, 6, 7, 8, 9, 10, 11, 12, 13, 14, or 15 days) after seeding of the plants. In particular embodiments, the inoculant composition described herein is applied to the legume plants once the plants have developed roots (e.g., roots with functional root hairs) after seeding of the plants.

The inoculant composition may further include at least one of an herbicide, an herbicide safener, a surfactant, a fungicide, a pesticide, a nematicide, a plant activator, a synergist, a plant growth regulator, an insect repellant, an acaricide, a molluscicide, or a fertilizer. The inoculant composition may also include one or more of: a surface-active agent, an inert carrier, a preservative, a humectant, a feeding stimulant, an attractant, an encapsulating agent, a binder, an emulsifier, a dye, a UV protective, a buffer, a flow agent, a fertilizer, micronutrient donors, or other preparations that influence plant growth. The inoculant composition can also include one or more agrochemicals including: herbicides, insecticides, fungicides, bactericides, nematicides, molluscicides, acaracides, plant growth regulators, harvest aids, and fertilizers, which can also be combined with carriers, surfactants, or adjuvants as appropriate for the agrochemical. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers.

Surface-active agents that can be used in the inoculant composition described herein include anionic compounds such as a carboxylate of, for example, a metal; carboxylate of a long chain fatty acid; an N-acylsarcosinate; mono- or di-esters of phosphoric acid with fatty alcohol ethoxylates or salts of such esters; fatty alcohol sulfates such as sodium dodecyl sulfate, sodium octadecyl sulfate or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitar fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials or inert carriers that can be used include, but are not limited to, inorganic minerals such as kaolin, phyllosilicates, carbonates, sulfates, phosphates, or botanical materials such as cork, powdered corncobs, peanut hulls, rice hulls, and walnut shells. Herbicides that can be used in the inoculant composition include compounds that kill or inhibit growth or replication of undesired plants, typically a subset of plants that is distinct from the desired plant or crop. There are several modes of action: ACCase inhibition, carotenoid biosynthesis inhibition, cell wall synthesis inhibition, ALS inhibition, ESP synthase inhibition, glutamine synthase inhibition, HPPD inhibition, microtubule assembly inhibition, PPO inhibition, etc. Examples of commercially available herbicides include One-Time®, MSMA, Corvus®, Volunteer®, Escalade®, Q4®, Raptor®, Acumen®, Sencor®, Bullet®, TopNotch®, Valor®, PastureGard®, glycophosate (Roundup®), DSMA, Break-Up®, Hyvar®, Barricade®, etc. Herbicides can be mixed with “herbicide safeners” to reduce general toxicity of the herbicide, as described, e.g., in Riechers et al. (2010) Plant Physiol. 153:3.

Pesticides (e.g., nematicides, molluscicides, insecticides, miticide/acaricides) can be used to kill or reduce the population of undesirable pests affecting the plant. Pesticides can also be used with repellants or pheromones to disrupt mating behavior. Insectides are directed to insects, and include, e.g., those of botanical origin (e.g., allicin, nicotine, oxymatrine, jasmolin I and II, quassia, rhodojaponin III, and limonene), carbamate insecticides (e.g., carbaryl, carbofuran, carbosulfan, oxamyl, nitrilacarb, CPMC, EMPC, fenobucarb), fluorine insecticides, formamidine insecticides, fumigants (e.g., ethylene oxide, methyl bromide, carbon disulfide), chitin synthesis inhibitors, macrocyclic lactone insecticides, neonicotinoid insecticides, organophosphate insectides, urea and thiourea insectides, etc. Nematicides affect nematodes, and include, e.g., organophosphorus nematicides (e.g., diamidafos, fosthiazate, heterophos, phsphamidon, triazophos), fumigant nematicides (e.g., carbon disulfide, methyl bromide, methyl iodide), abamectin, carvacrol, carbamate nematicides (e.g., benomyl, oxamyl), etc. Molluscicides are directed to slugs and snails, and include, e.g., allicin, bromoacetamide, thiocarb, trifenmorph, fentin, copper sulfate, etc. Many pesticides target more than one type of pest, so that one or two can be selected to target insects, mollusks, nematodes, mitogens, etc.

Fertilizers typically provide macro- and micronutrients in a form that they can be utilized by the plant, or a plant-associated organism. These include, e.g., nitrogen, phosphorus, potassium, sulfur, calcium, potassium, boron, chlorine, copper, iron, manganese, molybdenum, zinc, nickel, and selenium. Fertilizers are often tailored to specific soil conditions or for particular crops or plants. Fertilizers that can be used in the inoculant composition include naturally-occurring, modified, concentrated and/or chemically synthesized materials, e.g., manure, bone meal, compost, fish meal, wood chips, etc., or can be chemically synthesized, UAN, anhydrous ammonium nitrate, urea, potash, etc. Suppliers include Scott®, SureCrop®, BCF®, RVR®, Gardenline®, and many others known in the art.

Fungicides are compounds that can kill fungi or inhibit fungal growth or replication. Fungicides that can be used include contact, translaminar, and systemic fungicides. Examples include sulfur, neem oil, rosemary oil, jojoba, tea tree oil, Bacillus subtilis, Ulocladium, cinnamaldehyde, etc.

### IV. Methods

The disclosure provides methods of infecting a legume plant and/or increasing the yield of a legume plant by (a) providing a population of light-activated, nitrogen-fixing bacteria by illuminating a population of nitrogen-fixing bacteria with a light having a wavelength of between 350 and 750 nm and an intensity of between 0.1 and 200 μmol·m⁻²·s⁻¹ for a period of between 1 second and 24 hours, and (b) delivering to the legume plant the population of light-activated, nitrogen-fixing bacteria. The present disclosure is directed to the concept that illumination of nitrogen-fixing bacteria (e.g., Rhizobium leguminosarum) could result in an enhancement in the capacity of the bacteria to infect legume plants and consequently improve legume agriculture.

Legume crops are routinely sprayed with nitrogen-fixing bacteria that infect the plants and live inside the plant roots (i.e., root nodules) to provide the plants with fertilizing compounds that they produce from nitrogen in the air. In return, the plants provide the bacteria with the nutrients they need to survive. As shown in the Examples section, inoculation of legume plants with blue light-treated bacteria led to a significant improvement of plant development over inoculation with non-illuminated bacteria. Pre-illumination of the bacteria before inoculation of the bacteria to the growing crop greatly improves their capacity to infect the plant roots.

Furthermore, the methods of infecting a legume plant and/or increasing the yield of a legume plant as described herein comprise the step of delivering the population of light-activated, nitrogen-fixing bacteria (e.g., Rhizobium leguminosarum) to the legume plant after the plant has already developed a root with functional root hairs and the population of light-activated, nitrogen-fixing bacteria are able to infect the root of the legume plant. Delaying the timing of inoculation until a plant has germinated and has generated a root system provides immediate opportunities for the bacteria to infect the roots of the plant, thus, increasing the survival rate of the bacteria. In particular embodiments of the methods described herein, the population of light-activated, nitrogen-fixing bacteria are delivered to the legume plants at least 24 hours (e.g., at least 36 hours, at least 48 hours, at least 60 hours, at least 72 hours, at least 84 hours, at least 96 hours, or at least 108 hours, at least 120 hours, at least 132 hours, or at least 144 hours) after the legume plant seeds have been planted. As shown in the Examples section, legume plants inoculated with blue-light illuminated bacteria at six days after the seeding of the plants, at which time the plants had developed roots, were able to generate more functional nodules than plants that were inoculated with blue-light illuminated bacteria at the same time as the seeding of the plants or plants that were inoculated with non-illuminated bacteria (FIG. 6). The blue-light illuminated bacteria induced the generation of functional nodules (i.e., red or pink nodules that contain leghemoglobin) that can house the inoculated bacteria which convert free nitrogen to ammonia for the plant to utilize for its development. In some embodiments, the methods described herein result in an increased number of red or pink nodule formation compared to the number of red or pink nodules formed in a legume plant infected by a population of nitrogen-fixing bacteria that is not light activated.

In some embodiments of methods of increasing the yield of a legume plant as described herein, the yield of the legume plant is at least 6% greater (e.g., at least 10%, at least 12%, at least 14%, at least 16%, at least 18%, at least 20%, at least 22%, at least 24%, at least 26%, at least 28%, at least 30%, at least 32%, at least 34%, at least 36%, at least 38%, at least 40%, at least 42%, at least 44%, at least 46$%, at least 48%, or at least 50%) than the yield of a legume plant that is infected by a population of nitrogen-fixing bacteria that is not light activated. In some embodiments, the yield of the legume plant is at least 6% greater (e.g., at least 10%, at least 12%, at least 14%, at least 16%, at least 18%, at least 20%, at least 22%, at least 24%, at least 26%, at least 28%, at least 30%, at least 32%, at least 34%, at least 36%, at least 38%, at least 40%, at least 42%, at least 44%, at least 46$%, at least 48%, or at least 50%) than the yield of a legume plant that is infected by a population of nitrogen-fixing bacteria that is not light activated or a population of light-activated, nitrogen-fixing bacteria at a time before the legume plant has generated any root system.

In some embodiments of methods of infecting a legume plant and/or increasing the yield of a legume plant as described herein, the population of light-activated, nitrogen-fixing bacteria may be generated by illuminating a population of nitrogen-fixing bacteria with a light (e.g., an LED light) having a wavelength of between 350 and 750 nm (e.g., between 350 and 700 nm, between 350 and 650 nm, between 350 and 600 nm, between 350 and 550 nm, between 350 and 500 nm, between 350 and 450 nm, between 350 and 400 nm, between 400 and 750 nm, between 450 and 750 nm, between 500 and 750 nm, between 550 and 750 nm, between 600 and 750 nm, between 650 and 750 nm, between 700 and 750 nm, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, or 750 nm). In some embodiments, the bacteria may be illuminated with a light having a wavelength of between 400 and 500 nm (e.g., between 410 and 500 nm, between 420 and 500 nm, between 430 and 500 nm, between 440 and 500 nm, between 450 and 500 nm, between 460 and 500 nm, between 470 and 500 nm, between 480 and 500 nm, between 490 and 500 nm, between 400 and 490 nm, between 400 and 480 nm, between 400 and 470 nm, between 400 and 460 nm, between 400 and 450 nm, between 400 and 440 nm, between 400 and 430 nm, between 400 and 420 nm, or between 400 and 410 nm). In particular embodiments, the bacteria may be illuminated with a light having a wavelength of between 445 and 455 nm (e.g., 445, 446, 447, 448, 449, 450, 451, 452, 453, 454, or 455 nm). In particular embodiments, the bacteria may be illuminated with a light having a wavelength of 450 nm.

In some embodiments of the methods described herein, the intensity and duration of light (e.g., light having a wavelength of between 445 and 455 nm, e.g., 450 nm) used to generate the population of light-activated, nitrogen-fixing bacteria may be between 0.1 and 200 μmol·m⁻²'s⁻¹ (e.g., between 0.1 and 150 μmol·m⁻²·s⁻¹, between 0.1 and 100 μmol·m² between 0.1 and 50 μmol·m⁻²·s⁻¹, between 0.1 and 10 μmol·m⁻²·s⁻¹, between 0.1 and 1 μmol·m⁻²·s⁻¹, between 0.1 and 0.5 μmol·m⁻²·s⁻¹, between 0.5 and 200 μmol·m⁻²·s⁻¹, between 1 and 200 μmol·m⁻²·s⁻¹, between 10 and 200 μmol·m⁻²·s⁻¹, between 50 and 200 μmol·m⁻²·s⁻¹, between 100 and 200 μmol·m⁻²·s⁻¹, or between 150 and 200 μmol·m²·s⁻¹) for a period of between 1 second and 24 hours (e.g., between 1 second and 20 hours, between 1 second and 15 hours, between 1 second and 10 hours, between 1 second and 5 hours, between 1 second and 1 hour, between 1 second and 30 minutes, between 1 second and 20 minutes, between 1 second and 10 minutes, between 1 second and 1 minute, between 1 second and 30 seconds, between 1 second and 10 seconds, between 1 second and 5 seconds, between 30 seconds and 24 hours, between 1 minute and 24 hours, between 10 minutes and 24 hours, between 24 minutes and 24 hours, between 30 minutes and 24 hours, between 1 hour and 24 hours, between 5 hours and 24 hours, between 10 hours and 24 hours, between 15 hours and 24 hours, or between 20 hours and 24 hours).

Once the legume plants have developed roots with functional root hairs for the bacteria to infect, the population of light-activated, nitrogen-fixing bacteria may be delivered to the legume plants using an irrigation system (e.g., a drip irrigation system). Examples of irrigation systems are described further herein.

Aside from the benefits of increased bacterial inoculation efficiency when the plants are inoculated after they have developed functional root hairs, as well as increased plant yield, the methods described herein also increase the number of nodules containing leghemoglobin formed on the root of the legume plants compared to the number of nodules containing leghemoglobin formed on the root of legume plants infected by a population of nitrogen-fixing bacteria that is not light activated. Moreover, in some embodiments, the methods also result in a greater number of leghemoglobin per nodule on the root of the legume plants compared to the number of leghemoglobin per nodule on the root of a legume plant infected by a population of nitrogen-fixing bacteria that is not light activated. In further embodiments, the methods also result in an increased number of nodule formation compared to the number of nodules formed in a legume plant infected by a population of nitrogen-fixing bacteria that is not light activated.

Examples of nitrogen-fixing bacteria that may be used in methods described herein include, but are not limited to, bacteria in a genus selected from the group consisting of Allorhizobiaum, Aminobacter, Azorhizobium, Bradyrhizobium (e.g., Bradyrhizobium japonicum), Devosia, Ensifer, Mesorhizobium, Methylobacterium, Microvirga, Ochrobactrum, Phyllobacterium, Rhizobium, Shinella, or Sinorhizobium (e.g., Sinorhizobium meliloti). In particular embodiments, the nitrogen-fixing bacteria used in methods described herein are Rhizobium (e.g., R. aggregatum, R. alamii, R. alkalisoli, R. borbori, R. cellulosilyticum, R.cireri, R. daejeonense, R. endophyticum, R. etli, R. fabae, R. fredii, R. galegae, R. gallicum, R. giardinii, R. hainanense, R. halophytocola, R. herbae, R. huakuii, R. huautlense, R. indigoferae, R. japonicum, R. larrymoorei, R. leguminosarum, R. leucaenae, R. loessense, R. loti, R. lupini, R. lusitanum, R. mediterraneum, R. melioti, R. misosinicum, R. miluonense, R. mongolense, R. multihospitium, R. oryze, R. petrolearium, R. phaseoli, R. pisi, R. pseudoryzae, R. pusense, R. radiobacter, R. rhizogenese, R. rosettiformans, R. rubi, R. selenitireducens, R. skierniewicense, R. soli, R. sullae, R. taibaishanense, R. tianshanense, R. tibeticum, R. trifolii, R. sphaerophysae, R. tropici, R. grahamii, R. mesoameicanum, R. nepotum, R. tubonense, R. undicola, R. vallis, R. vignae, R. vitis, or R. yanglingense; e.g., R. leguminosarum).

In some embodiments, the nitrogen-fixing bacteria may be a wild-type bacteria that express a LOV domain. In other embodiments, the nitrogen-fixing bacteria may be genetically engineered to expression a LOV domain.

Examples of legume plants that may benefit from the methods described herein include, but are not limited to, peas, soybeans, alfalfa, clover, vetch, mung bean, vigna, cowpea, trefoil, lupine, peanuts, fava beans, chickpeas, lentils, lupin beans, mesquite, carob, and tamarind.

### V. Engineered Bacteria

Nitrogen-fixing bacteria may be engineered to comprise an expression cassettes for expressing a photoreceptor (e.g., a LOV domain). In some embodiments, engineered, nitrogen-fixing bacteria may be generated to contain a complete or partial sequence of a polynucleotide that encodes a LOV domain. An expression vector comprising a LOV domain coding sequence driven by a promoter may be introduced into the genome of the bacteria host by a variety of conventional techniques. For example, the DNA construct may be introduced directly into the genomic DNA of the bacteria using techniques such as electroporation and microinjection, or the DNA construct can be introduced directly using ballistic methods, such as DNA particle bombardment. Alternatively, the DNA construct may be introduced into a viral host vector. The virulence functions of the viral host vector will direct the insertion of the construct into the bacterial DNA when the bacteria are infected by the virus. While transient expression of the constitutively active LOV domain is encompassed by the disclosure, generally, expression of a construct will be from insertion of expression cassettes into the bacterial genome, e.g., such that at least some bacterial offspring also contain the integrated expression cassette.

Microinjection techniques for insertion of an expression cassette into a host genome are well-known in the art. For example, the introduction of DNA constructs using polyethylene glycol precipitation is described in Paszkowski et al. EMBO J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Engineered bacterial cells derived by any of the above transformation techniques can be cultured to regenerate bacterial cells that possess the transformed genotype and thus the desired phenotype, e.g., expression of LOV domain and light sensitivity. The expression cassettes and other constructs can be used to engineer essentially any bacteria to express a LOV domain

Expression Cassettes

In some embodiments, an expression cassette comprising a polynucleotide encoding a LOV domain may be introduced into bacterial cells to generate engineered bacteria expressing a LOV domain. In some embodiments, a promoter may be operably linked to the polynucleotide encoding the LOV domain. The promoter may be heterologous to the polynucleotide. In some embodiments, the promoter may be inducible. In some embodiments, the polynucleotide encoding a LOV domain may be expressed constitutively. Examples of environmental conditions that may affect transcription by inducible promoters include, but are not limited to, anaerobic conditions, elevated temperature, and the presence of light. The vector comprising the polynucleotide sequence may include a marker gene that confers a selectable phenotype on bacterial cells. For example, the marker may encode biocide resistance, particularly antibiotic resistance, such as resistance to kanamycin, G418, bleomycin, hygromycin, or herbicide resistance, such as resistance to chlorosluforon or Basta. In some embodiments, the polynucleotide encoding the LOV domain may be expressed recombinantly in bacterial cells. A variety of different expression constructs, such as expression cassettes and vectors suitable for transformation of bacterial cells, can be prepared. Techniques for transforming a wide variety of bacterial species are well-known in the art.

Promoters

In some embodiments, a fragment can be employed to direct expression of a polynucleotide encoding a LOV domain transformed into bacterial cells. The term “constitutive regulatory element” means a regulatory element that confers a level of expression upon an operatively linked nucleic molecule that is relatively independent of the cell type in which the constitutive regulatory element is expressed. Promoters that drive expression continuously under physiological conditions are referred to as “constitutive” promoters and are active under most environmental conditions and states of development or cell differentiation.

Alternatively, a promoter may direct expression of the polynucleotide encoding a LOV domain under the influence of changing environmental conditions or developmental conditions. Examples of environmental conditions that may affect transcription by inducible promoters include anaerobic conditions, elevated temperature, or the presence of light. Such promoters are referred to herein as “inducible” promoters. In some embodiments, an inducible promoter is one that is induced by one or more environmental stressors. In some embodiments, promoters may be inducible upon exposure to chemicals or reagents that may be applied to the plant, such as herbicides or antibiotics. Some examples of inducible regulatory elements include, e.g., copper-inducible regulatory elements (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Roder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)).

### VI. Devices

Also encompassed in the disclosure is a device that can generate a light-activated, nitrogen-fixing bacterial (e.g., Rhizobium) culture and deliver the light-activated bacterial (e.g., Rhizobium) culture to a legume plant. The device may include an illumination system that functions to activate the bacteria in the nitrogen-fixing bacterial culture, in which the bacteria in the culture comprise a LOV domain. The device may also include a delivery system that functions to provide the light-activated, nitrogen-fixing bacterial (e.g., Rhizobium) culture to the legume plant.

The illumination system in the device may comprise a light source that is able to generate light of varying wavelengths, e.g., a wavelength of between 350 and 750 nm (e.g., between 350 and 700 nm, between 350 and 650 nm, between 350 and 600 nm, between 350 and 550 nm, between 350 and 500 nm, between 350 and 450 nm, between 350 and 400 nm, between 400 and 750 nm, between 450 and 750 nm, between 500 and 750 nm, between 550 and 750 nm, between 600 and 750 nm, between 650 and 750 nm, between 700 and 750 nm, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, or 750 nm). In particular embodiments, the illumination system generates a light having a wavelength of between 400 and 500 nm (e.g., between 445 and 455 nm; e.g., 450 nm).

The illumination system may include additional controls that allow the adjustment of the intensity of the light, as well as the duration of light generation. For example, a light of strong intensity (e.g., between 100 and 200 μmol·m²·s⁻¹; e.g., 120, 140, 160, 180, or 200 μmol·m⁻²·s⁻¹) may only be needed for a brief period of time (e.g., between 1 second and 1 hour; e.g., 30 seconds, 1 minute, 10 minutes, 20 minutes, 30 minutes, 40 minutes, 50 minutes, or 1 hour) in order to sufficiently activate the bacteria (e.g., Rhizobium (e.g., Rhizobium leguminosarum)). In other embodiments, a light of relatively weak intensity (e.g., between 0.1 and 100 μmol·m⁻²·s⁻¹; e.g., 0.5, 1, 10, 20, 40, 60, 80, or 100 μmol·m⁻²·s⁻¹) may be needed for a longer period of time (e.g., between 1 hour and 24 hours; e.g., 2, 4, 6, 8, 10, 12, 14, 16, 18, 20, 22, or 24 hours) in order to sufficiently activate the bacteria (e.g., Rhizobium (e.g., Rhizobium leguminosarum)). One of skill in the art has the knowledge and capabilities to adjust the intensity and duration of the light generation from the illumination system in the device in order to achieve the most efficient bacteria activation, such that inoculation of the light-activated bacteria to the legume plants results in high infection efficiency, as well as increased yield of the plants, increased number of nodule formation, and/or increased number of leghemoglobin per nodule on the root of the legume plant.

In some embodiments, the distance between the nitrogen-fixing bacterial (e.g., Rhizobium) culture and the light source may be adjusted to prevent overheating or scorching. In some embodiments, the light source in the illumination system is an LED light.

The device may also include a delivery system that provides the light-activated, nitrogen-fixing bacterial (e.g., Rhizobium) culture to the legume plant. In some embodiments, the delivery system may be an irrigation system. In some embodiments, the irrigation system may be drip irrigation, surface irrigation, micro-irrigation, sprinkler irrigation, center pivot irrigation, movable irrigation, or subirrigation. In particular embodiments, the irrigation system in the device is drip irrigation. In a drip irrigation, the light-activated, nitrogen-fixing bacterial (e.g., Rhizobium) culture may be provided to legume plants drop by drop at or near the position of the roots of the plants. In some embodiments, the irrigation system may comprise a piped network covering the field of legume plants to distribute the light-activated, nitrogen-fixing bacterial (e.g., Rhizobium) culture evenly to the plants. In some embodiments, the piped network may be underground in order to deliver the bacteria more directly to the roots of the plants. The delivery system may further include controls to adjust the pressure, amount, and/or timing of bacteria culture delivery.

### EXAMPLES

### Example 1—Effects of Blue-Light Irradiated Bacterial Cells on Fava Bean Plants

The purpose of the experiment was the following: first, to determine whether photoactivation of Rhizobium leguminosarum nodulation capability would in any way improve the nodulation, growth, and final seed yield of fava beans (Vicia faba L.); second, to determine whether inoculation of fava beans not at the time of seed planting but several days later when a primary root had already emerged might in any way improve fava bean nodulation, growth, and final seed yield. Under the growth conditions, the results indicated the following: irradiation of Rhizobium leguminosarum to activate its capacity to form nodules prior to inoculation was effective either time in increasing fava-bean seed yield over seed yield from plants in which the inoculated bacteria had been held in the dark; second, inoculation after the fava bean primary roots had emerged was significantly more effective in increasing nodulation and seed yield than inoculation at the time of planting regardless of light treatment.

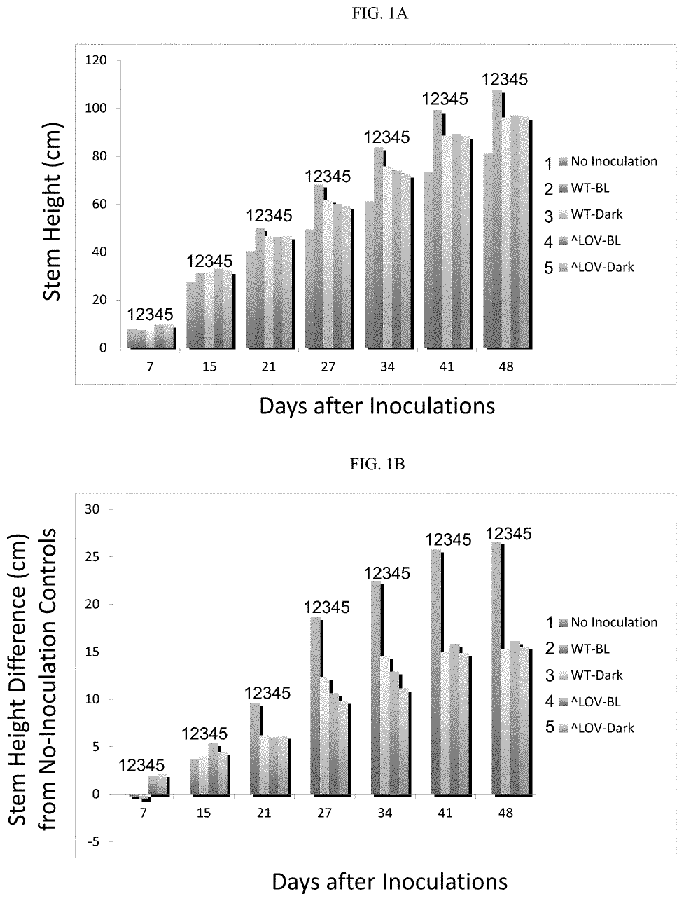

Rhizobium leguminosarum cells were irradiated with blue light (BL) and inoculated into the soil of fava bean seeds six days after the fava bean seeds were planted. As shown in FIGS. 1A and 1B, fava bean plants inoculated with blue-light irradiated Rhizobium leguminosarum cells had accelerated elongation growth. \hat{ }LOV indicates the bacteria that have their bacterial photoreceptor LOV domain inactivated.

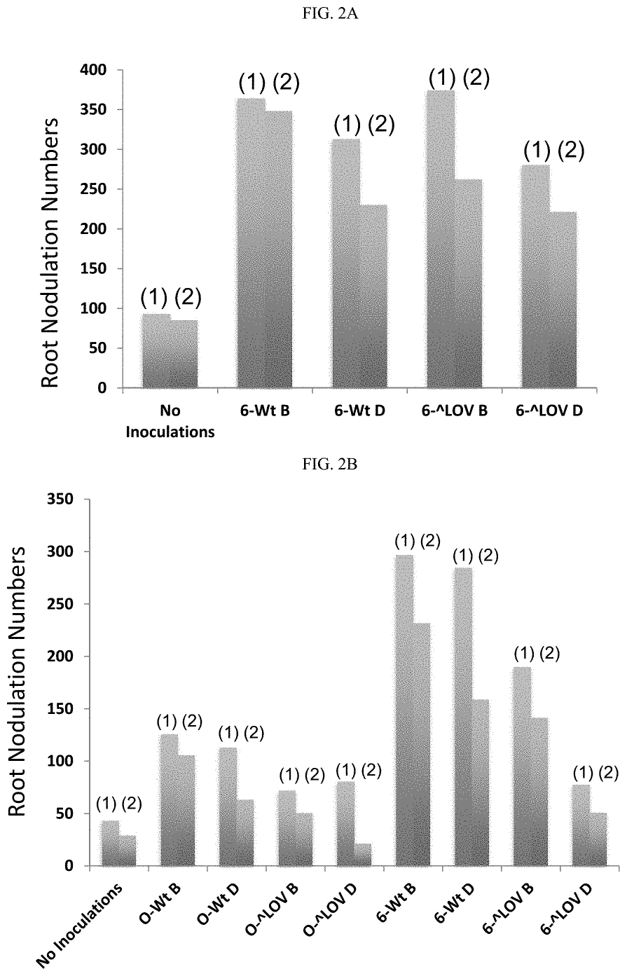

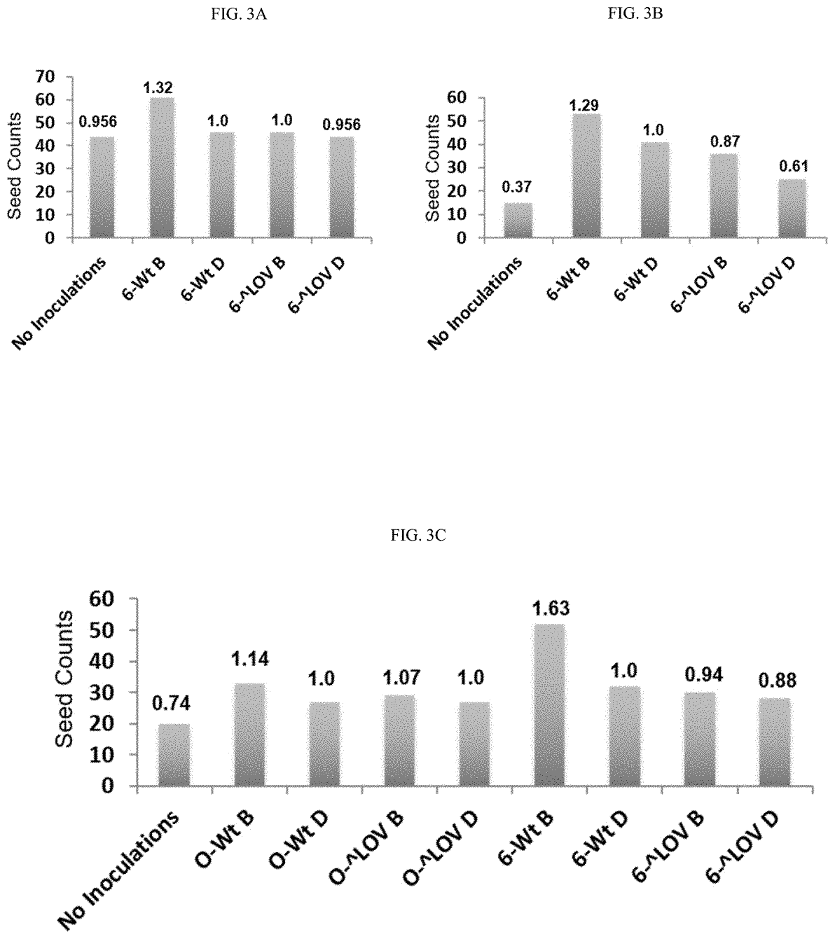

Moreover, FIG. 2A shows the nodulation numbers of five to six week old fava bean plants that were inoculated with Rhizobium leguminosarum cells either irradiated with blue light or kept in the dark at six days after the seeds were planted. (1) indicates the total number of nodules and (2) indicates the number of red or pink nodules that contain leghemoglobin and are the only functional nodules. Each bar includes the nodule counts from three plants. For \hat{ }LOV B, although the bacteria were supposed to have their photoreceptor LOV domain inactivated, the cultures clearly contained wild-type bacteria as indicated by the nodulation responses observed. FIG. 2B further shows a comparison of the nodule numbers of fava beans that were inoculated with Rhizobium leguminosarum cells at 0 days following seed planting and at 6 days following seed planting.

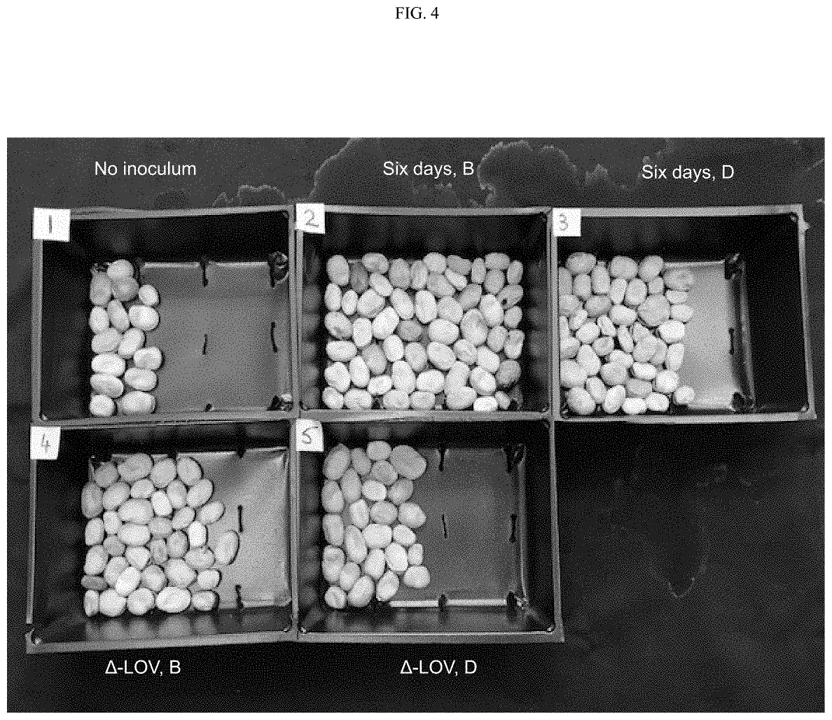

FIGS. 3A-3C demonstrate that fava bean seed yield increased when the plants were inoculated with Rhizobium leguminosarum cells irradiated with blue light either at 0 days following seed planting and at 6 days following seed planting. Each bar represents the yield from eight plants. The number above each bar indicates the yield relative to the dark control.

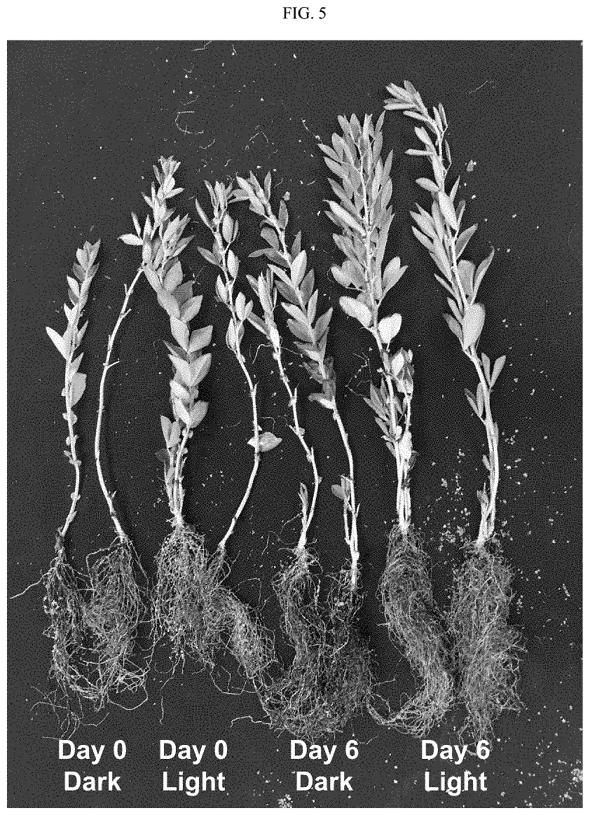

FIG. 4 is a photograph showing the yields of fava beans that had no bacteria inoculation (No inoculum), inoculated with Rhizobium leguminosarum cells irradiated with blue light at six days after seed planting (Six days, B), inoculated with Rhizobium leguminosarum cells kept in the dark at six days after seed planting (Six days, D), inoculated with blue light-irradiated Rhizobium leguminosarum cells having an inactivated LOV domain at six days after seed planting (A-LOV, B), and inoculated with dark Rhizobium leguminosarum cells having an inactivated LOV domain at six days after seed planting (A-LOV, D). Each box contained the yields from eight fava bean plants.

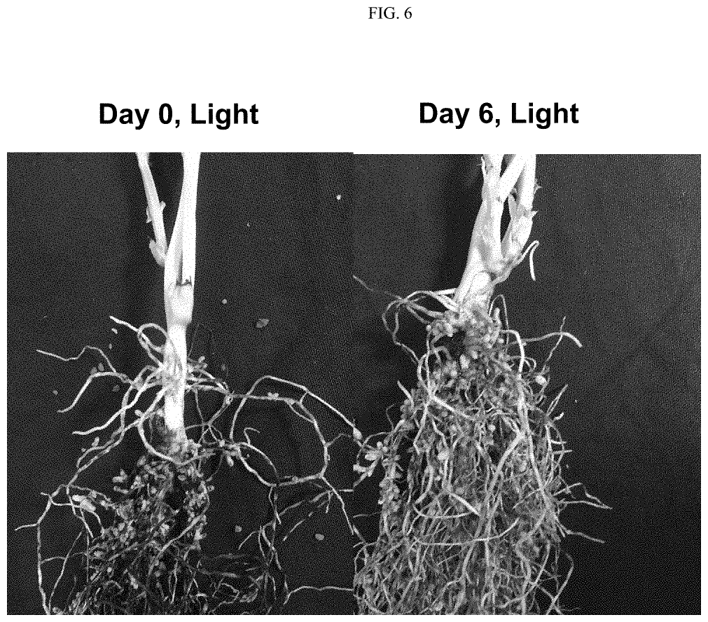

Further, the difference of functional nodule formation on plants inoculated with Rhizobium leguminosarum cells irradiated with blue light and Rhizobium leguminosarum cells kept in the dark is shown in FIGS. 5 and 6. Fava bean plants inoculated at six days after seed planting with blue light-irradiated Rhizobium leguminosarum cells developed more functional nodules (red nodules) compared to plants inoculated with Rhizobium leguminosarum cells kept in the dark (FIG. 6).

### Example 2—Functional Nodules

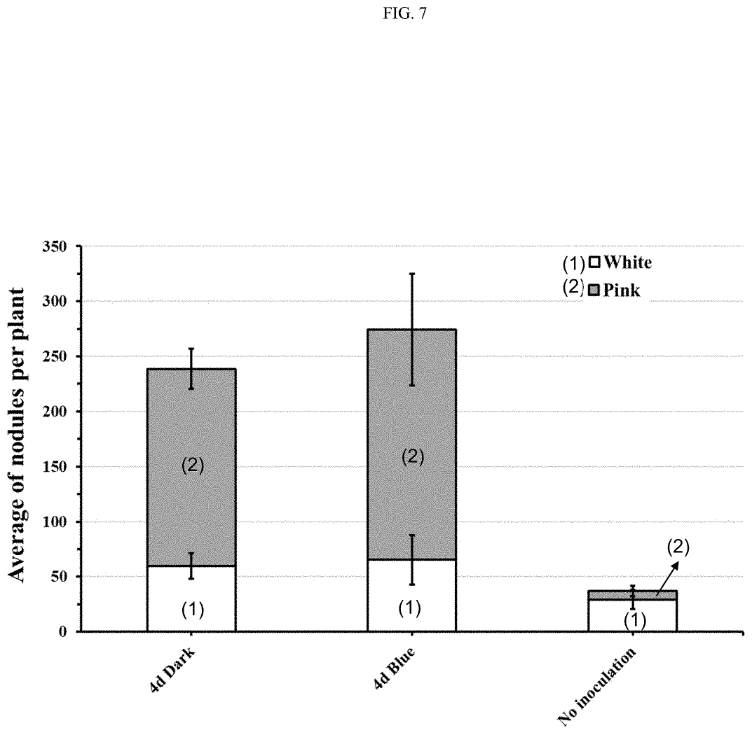

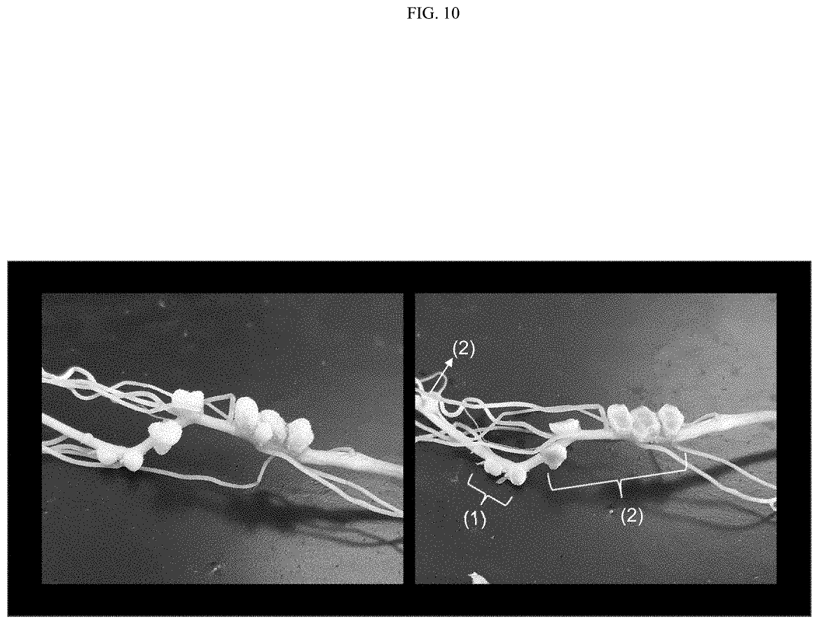

FIG. 10 shows functional nodules (2) containing leghemoglobin on a root system inoculated with bacteria treated under blue light. Further, FIG. 7 also shows that plants inoculated with bacteria exposed to blue light and watered 4 days prior the inoculation showed higher production of pink nodules than plants inoculated with bacteria exposed to no light (dark condition). FIG. 7 shows a representation of the average number of nodules per plant found on its root system. The plants were watered 4 days before the bacteria inoculation (R. leguminosarum after 8 hours of exposition to blue light (4d Blue (4DB)) or kept in continuous dark (4d Dark (4DD))) or no inoculation (NO). The graph presents the total of nodules counting per plant (average from 10 plants for 4d Blue; average from 12 plants for 4d Dark; aver from 14 plants for No Inoculation) with the number of functional nodules characterized by their pink pigment (2) and white nodules (1). Error bars were calculated by SEM. Further, Table 1 below shows that under the 4d Blue condition, about 76.1% of the nodules produced are pink when about 74.6% of the nodules are pink under 4d Dark condition. The number of nodules produced on the root system of plants inoculated by bacteria exposed to blue light (4DB) is about 14.4% more than the number of nodules produced on the root system of plants inoculated by bacteria kept in the dark (4DD).

TABLE 1

| | Nodules/plant | | |
|---|---|---|---|
| | Total | White | Pink |
| 4DD | 239.8 ± 23.99 | 59.75 ± 11.68 | 178.8 ± 18.30 |
| 4DB | 274.3 ± 54.91 | 65.30 ± 22.35 | 209.0 ± 50.54 |
| NO | 36.93 ± 11.71 | 29.21 ± 8.686 | 7.714 ± 4.866 |

### Example 3—Total Pod and Pea Production

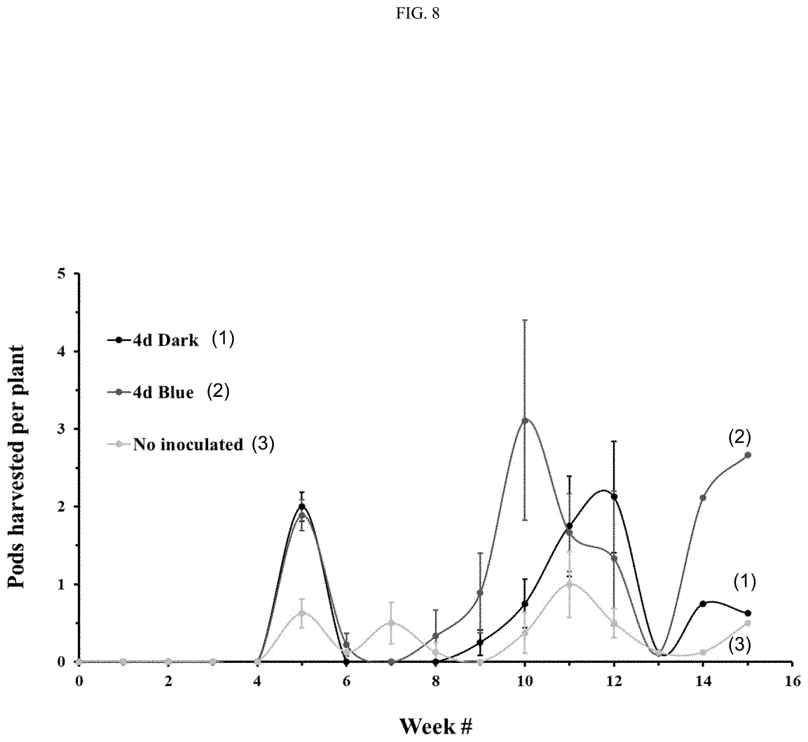

Plants treated with the bacteria exposed to blue light showed a higher production of mature pods than the plants treated with the bacteria exposed to continuous dark, as shown in FIG. 8. FIG. 8 and Table 2 below are representations of the number of mature pods harvested on plants watered 4 days before the bacteria inoculation (R. leguminosarum after 8 hours of exposition of blue light (4d Blue (4DB)) or kept in continuous dark (4d Dark (4DD))) or no inoculation (NO). The numbers present the average number of mature pods harvested per plant (average from 9 plants for 4d Blue; average from 8 plants for 4d Dark; aver from 8 plants for No Inoculation) over 11 weeks. All mature pods were all collected once per week. Error bars were calculated by SEM. Table 2 shows the average number of pods harvested per plant over the 11 weeks of collection. FIG. 8 also shows that plants from the 4DB condition were continuously producing at a high level contrary to plants from the 4DD condition, which slowed down progressively. This observation is highlighted by the average number of pods harvested per plant (in total) (Table 2).

TABLE 2
Total Pods harvested/plant

| | |
|---|---|
| 4DD | 8.375 |
| 4DB | 14.33 |
| NO | 4 |

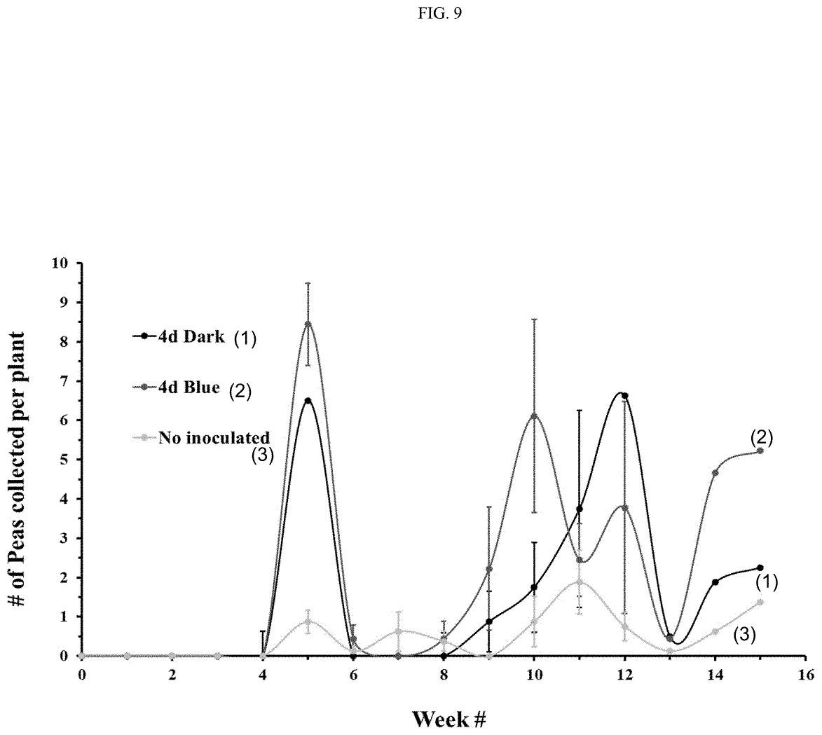

Plants treated with the bacteria exposed to blue light showed a higher production of peas than the plants treated with the bacteria exposed to continuous dark, as shown in FIG. 9. FIG. 9 and Table 3 below are representations of the number of peas harvested on plants watered 4 days before the bacteria inoculation (R. leguminosarum after 8 hours of exposition of blue light (4d Blue (4DB)) or kept in continuous dark (4d Dark (4DD))) or no inoculation (NO). The numbers present the average number of peas harvested per plant (average from 9 plants for 4d Blue; average from 8 plants for 4d Dark; aver from 8 plants for No Inoculation) over 11 weeks. Peas were collected from all the mature pods harvested once per week. Error bars were calculated by SEM. Table 3 shows the average number of peas harvested per plant over the 11 weeks of collection. Plants treated under the 4DB condition showed a notable increase of the number of peas collected (about 42% increase) than plants treated under the 4DD condition.

TABLE 3
Total Peas harvested/plant:

| | |
|---|---|
| 4DD | 24.13 |
| 4DB | 34.22 |
| NO | 7.625 |

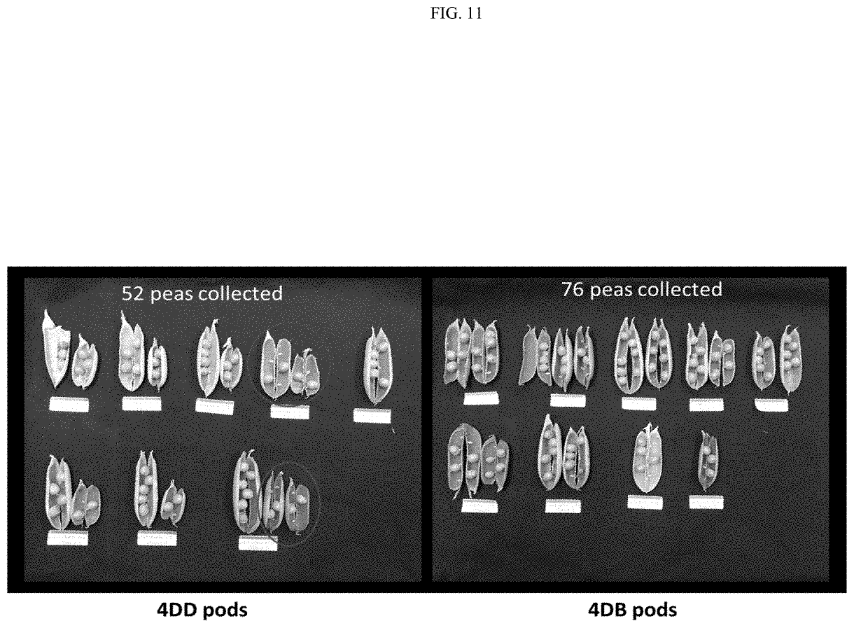

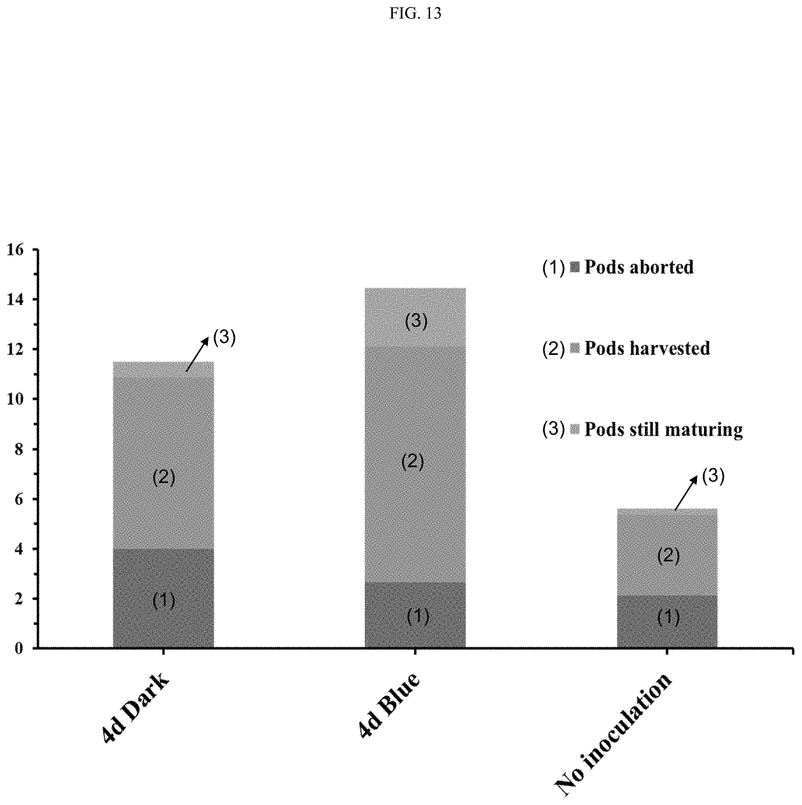

Moreover, FIG. 11 further shows that plants inoculated with light-activated bacteria Rhizobium produced more peas per pod on average. FIG. 11 includes images of open pods harvested from plants watered 4 days before the bacteria inoculation (R. leguminosarum after 8 hours of exposition of blue light (4d Blue, 4DB) or kept in continuous dark (4d Dark, 4DD)). Circles indicate the pods in the condition 4DD where a majority of peas was aborted in pods. FIG. 13 is a bar graph showing an average per plant of the portions of pods aborted (1), harvested (2), and still maturing (3) at the end of the 8 weeks of growth period. As shown in FIG. 13, plants treated with light-activated Rhizobium showed a decrease in the number of pods aborted.

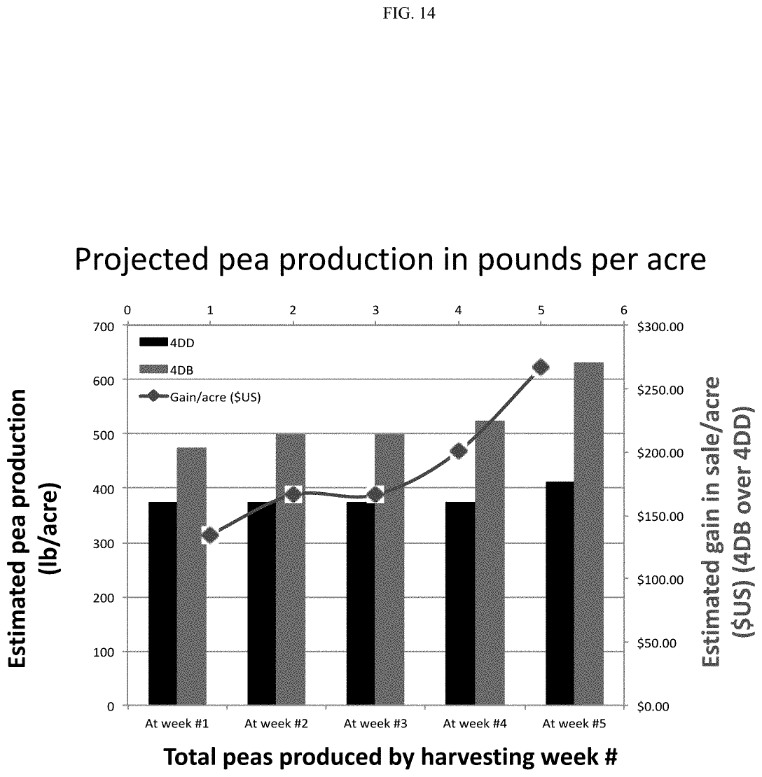

Furthermore, inoculating plants with bacteria treated under blue light condition is projected to increase plant yield. FIG. 14 shows projected weights of peas produced per acre. The estimated values were calculated based on the average of 30,000 plants planted per acre on a commercial farm. The increase in value ($US) projected for plants inoculated with bacteria treated under blue light condition (4DB) over plants inoculated with bacteria kept under the dark (4DD) was calculated based on a sale price of $5 per pound of harvested peas. No-I (no-inoculation control), 4DD (Dark-grown Rhizobium applied at 4 days after germination), and 4DB (Blue-light treated Rhizobium applied at 4 days after germination).

### Example 4—Chlorophyll Production

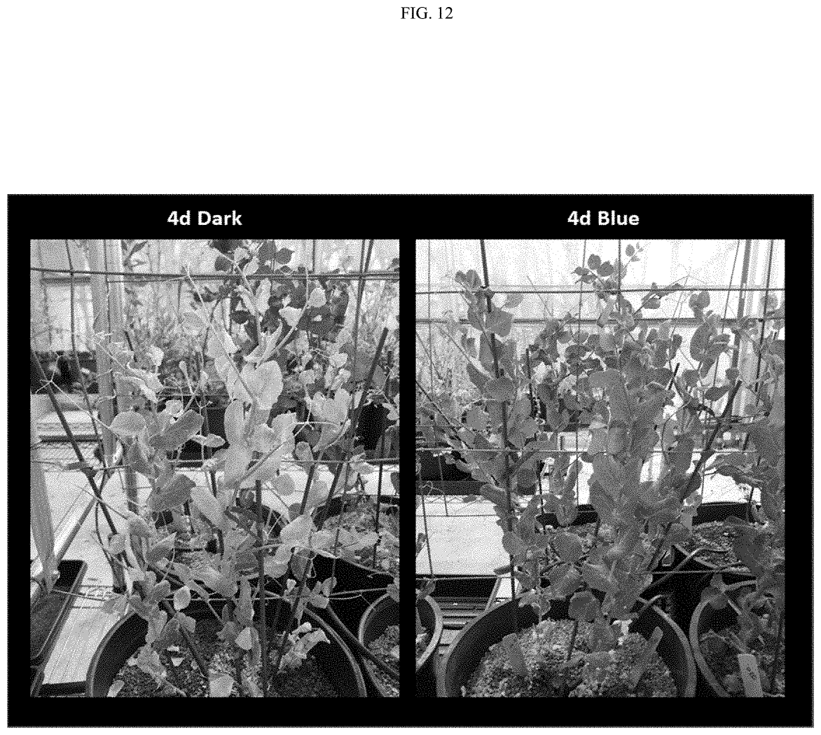

Plants treated with light-activated Rhizobium also displayed a good chlorophyll production compared to plants treated with Rhizobium left in the dark, after 75 days of growth period (FIG. 12). FIG. 12 shows images of three plants watered 4 days before the bacteria inoculation treated with R. leguminosarum after 8 hours of exposition of blue light (4d Blue, 4DB) (right) or treated with R. leguminosarum kept in continuous dark (4d Dark, 4DD) (left).

One or more features from any embodiments described herein or in the figures may be combined with one or more features of any other embodiment described herein in the figures without departing from the scope of the disclosure.

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

1. (canceled)

2. A method of increasing yield of a legume plant, comprising:
(a) providing a population of light-activated, nitrogen-fixing bacteria by illuminating a population of nitrogen-fixing bacteria with a light having a wavelength of between 350 and 750 nm and an intensity of between 0.1 and 200 μmol·m⁻²·s⁻¹ for a period of between 1 second and 24 hours, and
(b) delivering to the legume plant the population of light-activated, nitrogen-fixing bacteria,
wherein the legume plant comprises a root with functional root hairs, wherein the population of light-activated, nitrogen-fixing bacteria infects the root of the legume plant, and wherein the yield of the legume plant is at least 6% greater than the yield of a legume plant that is infected by a population of nitrogen-fixing bacteria that is not light activated.

3. The method of claim 2, wherein the population of nitrogen-fixing bacteria is illuminated with a light having a wavelength of between 400 and 500 nm.

4. The method of claim 3, wherein the wavelength is between 445 and 455 nm.

5. (canceled)

6. The method of claim 2, wherein the nitrogen-fixing bacteria comprise a light, oxygen, and voltage (LOV) domain.

7. The method of claim 6, wherein the nitrogen-fixing bacteria naturally express the LOV domain or are engineered to express the LOV domain.

8. (canceled)

9. The method of claim 2, wherein step (b) comprises delivering the light-activated, nitrogen-fixing bacteria to the legume plant through an irrigation system.

10. The method of claim 9, wherein the irrigation system is a drip irrigation system.

11. (canceled)

12. The method of claim 2, wherein the method results in a greater number of nodules containing leghemoglobin formed on the root of the legume plant compared to the number of nodules containing leghemoglobin formed on the root of a legume plant that is infected by a population of nitrogen-fixing bacteria that is not light activated.

13. The method of claim 2, wherein the method results in a greater number of leghemoglobin per nodule on the root of the legume plant compared to the number of leghemoglobin per nodule on the root of a legume plant that is infected by a population of nitrogen-fixing bacteria that is not light activated.

14. The method of claim 2, wherein the population of nitrogen-fixing bacteria are in a genus selected from the group consisting of Allorhizobiaum, Aminobacter, Azorhizobium, Bradyrhizobium, Devosia, Ensifer, Mesorhizobium, Methylobacterium, Microvirga, Ochrobactrum, Phyllobacterium, Rhizobium, Shinella, or Sinorhizobium.

15. (canceled)

16. The method of claim 14, wherein the population of nitrogen-fixing bacteria are R. aggregatum, R. alamii, R. alkalisoli, R. borbori, R. cellulosilyticum, R.cireri, R. daejeonense, R. endophyticum, R. etli, R. fabae, R. fredii, R. galegae, R. gallicum, R. giardinii, R. hainanense, R. halophytocola, R. herbae, R. huakuii, R. huautlense, R. indigoferae, R. japonicum, R. larrymoorei, R. leguminosarum, R. leucaenae, R. loessense, R. loti, R. lupini, R. lusitanum, R. mediterraneum, R. melioti, R. misosinicum, R. miluonense, R. mongolense, R. multihospitium, R. oryze, R. petrolearium, R. phaseoli, R. pisi, R. pseudoryzae, R. pusense, R. radiobacter, R. rhizogenese, R. rosettiformans, R. rubi, R. selenitireducens, R. skierniewicense, R. soli, R. sullae, R. taibaishanense, R. tianshanense, R. tibeticum, R. trifolii, R. sphaerophysae, R. tropici, R. grahamii, R. mesoameicanum, R. nepotum, R. tubonense, R. undicola, R. vallis, R. vignae, R. vitis, R. yanglingense, Bradyrhizobium japonicum, or Sinorhizobium meliloti.

17. (canceled)

18. (canceled)

19. The method of claim 2, wherein the legume plant is selected from the group consisting of peas, soybeans, alfalfa, clover, vetch, mung bean, vigna, cowpea, trefoil, lupine, peanuts, fava beans, chickpeas, lentils, lupin beans, mesquite, carob, and tamarind.

20. A method of infecting a legume plant with a light-activated, nitrogen-fixing Rhizobium culture, the method comprising:
(a) activating a nitrogen-fixing Rhizobium culture by illuminating the nitrogen-fixing Rhizobium culture with a light having a wavelength of between 350 and 750 nm and an intensity of between 0.1 and 200 μmol·m⁻²·s⁻¹ for a period of between 1 second and 24 hours, thereby creating a light-activated, nitrogen-fixing Rhizobium culture; and
(b) contacting a legume plant seed with the light-activated, nitrogen-fixing Rhizobium culture after the legume plant seed has developed at least one functional root hair.

21. The method of claim 20, wherein the nitrogen-fixing Rhizobium culture is illuminated with a light having a wavelength of between 400 and 500 nm.

22. The method of claim 20 or 21, wavelength is between 445 and 455 nm.

23. (canceled)

24. The method of claim 20, wherein one or more nitrogen-fixing bacteria in the nitrogen-fixing Rhizobium culture comprise a LOV domain.

25. The method of claim 24, wherein the one or more nitrogen-fixing bacteria naturally express the LOV domain or are engineered to express the LOV domain.

26. (canceled)

27. The method of claim 20, wherein the nitrogen-fixing Rhizobium culture is illuminated with an LED light.

28. The method of claim 20, wherein step (b) occurs at least 24 hours, at least 48 hours, or at least 72 hours after the legume plant seed has been planted.

29. (canceled)

30. (canceled)

31. The method of claim 20, wherein step (b) comprises providing the nitrogen-fixing Rhizobium culture to the legume plant seed via drip irrigation.

32. A device that generates a light-activated, nitrogen-fixing Rhizobium culture and delivers the light-activated Rhizobium culture to a legume plant, the device comprising:
(a) a light source configured to provide light to a nitrogen-fixing Rhizobium culture at a wavelength of between 350 and 750 nm and an intensity of between 0.1 and 200 μmol·m⁻²·s⁻¹ for a period of between 1 second and 24 hours; and
(b) a delivery system configured to provide the light-activated, nitrogen-fixing Rhizobium culture to the legume plant subsequent to the activation of the nitrogen-fixing Rhizobium culture by the light source in step (a).

33. The device of claim 32, wherein the light source is configured to provide light at a wavelength of between 400 and 500 nm.

34. The device of claim 33, wherein the light source is configured to provide light at a wavelength of between 445 and 455 nm.

35. (canceled)

36. The device of claim 32, wherein the delivery system comprises a drip irrigation system.

37.-40. (canceled)
